SEQUENCE LISTING (2 sequences)

SEQ ID NO: 1 - protein, 172 aa, Ovis aries
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 2 - protein, 172 aa, Artificial
  Recombinant IFNTau Based on Ovis aries Sequence
CYLSERLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

(19) United States
(12) Patent Application Publication
(10) Pub. No.: US 2007/0025963 A1
(43) Pub. Date: Feb. 1, 2007

(54) Methods for reduction of scar tissue formation

(76) Inventor: Chih-Ping Liu, San Francisco, CA (US)

Correspondence Address: PERKINS COIE LLP, P.O. BOX 2168, MENLO PARK, CA, 94026, US

(21) Appl. No.: 11/481,658

(22) Filed: Jul. 5, 2006

Related U.S. Application Data
(60) Provisional application No. 60/703,123, filed on Jul. 27, 2005.

(51) Int. Cl. A61K 38/21

(52) U.S. Cl. 424/85.4

(57) ABSTRACT

A method for reducing formation of scar tissue by administering interferon-tau (IFNτ) to a patient is described. Methods for preventing excessive scar formation, such as keloid formation, and for reducing formation of adhesions by administering IFNτ are also described. Interferon-tau is administered systemically or locally in an amount effective to alter the normal processes of scar formation, but without preventing wound healing.

### CROSS-REFERENCE TO RELATED APPLICATIONS

This application claims the benefit of U.S. Provisional Application No. 60/703,123, filed Jul. 27, 2005, incorporated herein by reference in its entirety.

### TECHNICAL FIELD

The present subject matter described herein relates to a method of reducing or preventing formation of scar tissue during wound healing. The subject matter described herein also relates to methods for the prevention of adhesions, excessive scar formation, and other types of abnormal proliferation of tissue during a wound-healing process by administering interferon-tau.

### BACKGROUND

Wounds caused by trauma or surgery are accompanied by an initial inflammatory response which is a natural response of the body and a first step of the wound healing process. The initial inflammatory response is followed by the formation of fibrous tissue, more commonly referred to as scar tissue, by proliferation of fibroblasts which produce collagen, mucopolysaccharides, and gylcosaminoglycans at the wound site. A certain amount of inflammation is required in the early healing stages in order to clear away the cellular and protein debris that accumulates at the wound to avoid infection and/or chronic inflammation. The second stage of wound healing involves a repair process which entails the influx and proliferation of fibrous tissue, due in part by the production of collagen and other substances by the fibroblasts, resulting in the formation of dense fibrous connective tissue that is visually seen as a scar.

The process of wound healing broadly comprises a regeneration phase and a repair phase, the differentiation between the two based on the resultant tissue. In regeneration, specialized tissues are replaced by the proliferation of surrounding undamaged specialized cells. In repair, lost tissue is replaced by granulation tissue which matures to form scar tissue. The repair phase involves the generation of the repair material, which for the majority of musculoskeletal injuries, involves the production of scar (collagen) material. Generation of repair material occurs fairly soon after injury, typically within 24-48 hours, and continues for a period of several weeks after injury, the time period depending in part on the amount of vasculature in the injured tissue. During this period, the bulk of the scar material is formed, with scar formation being evident and ultimately complete with a functional scar is achieved.

As mentioned above, inflammation is a normal and necessary prerequisite to healing. The inflammatory events involve both a vascular cascade of events and a cellular cascade of events. These occur in parallel and are significantly interlinked. The inflammatory cascade involves production of chemical mediators that make an active contribution to the healing process. For example, the cellular cascade involves emigration of neutrophils, monocytes, lymphocytes, eosinophils, basophils, to the wounded area and production of chemical mediators. The inflammatory response results in a vascular response, by production of a cellular and fluid exudate, with resulting edema. The course of the inflammatory response will depend upon the number of cells destroyed, the original causation of the process and the tissue condition at the time of insult.

Following the inflammation phase, the wound repair begins, with scar formation resulting. In some subjects, the scar tissue formation process results in what is referred to as hypertrophic or keloid scars. A keloid scar is a raised, firm, thickened red scar that exceeds the boundary of the injury and may grow for a prolonged period of time. A keloid scar occurs when the tissue response is out of proportion to the amount of scar tissue required for normal repair and healing. The increase in scar size is due to deposition of an increased amount of collagen into the tissue. Keloid development has been associated with different types of skin injury including surgery, ear piercing, laceration, burns, vaccination or inflammatory process. Common sites are earlobes and the upper trunk and extremities.

Scar formation is both a cosmetic problem and can in some cases be a medical problem. For example, scars on the face following an injury or surgery undesirable and can negatively impact a person. In some cases, keloid development occurs and a visible, undesirable scar results. Moreover, intra-abdominal adhesions results in a very significant morbidity and mortality in every surgery practice. Treatment of pelvic adhesions following surgery are often performed, and repeat surgery can greatly aggravate scarring.

There remains a need for a treatment to prevent scar formation, to reduce excessive scar formation and to prevent development of adhesions. Mechanical barriers are currently used to prevent adhesion formation, and these are only minimally effective clinically. Keloids have been treated with injection of corticosteroid into the scar, by laser therapy, and by administration of pharmacologic agents that interfere with collagen synthesis. Methods for improving the appearance of scars and for prevention excessive scarring and adhesions, without the inhibition of wound healing, are needed.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

In one aspect, a treatment method for reducing scar formation is provided by administering an effective amount of interferon-tau (IFNτ). The IFNτ is administered to reduce scar tissue formation and/or to prevent excessive scar formation, without preventing wound healing. The IFNτ can be administered systemically, locally, or topically, as needed.

In addition to the exemplary aspects and embodiments described above, further aspects and embodiments will become apparent by reference to the drawings and by study of the following descriptions.

### BRIEF DESCRIPTION OF THE DRAWINGS

FIGS. 1A-1C are graphs showing the IL-10 serum level, in pg/mL, in human patients suffering from multiple sclerosis and treated orally with IFNτ, as a function of time, in days, for patient groups I, II, and III treated daily with 0.2 mg IFNτ (FIG. 1A), 0.6 mg IFNτ (FIG. 1B), and 1.8 mg IFNτ (FIG. 1C) from days 1-29.

FIG. 1D is a graph showing the mean IL-10 serum level, in pg/mL, for the human patients in each of the test Groups I, II, and III treated daily with 0.2 mg IFNτ (diamonds, Group I), 0.6 mg IFNτ (squares, Group II), and 1.8 mg IFNτ (triangles, Group III) from days 1-29.

### BRIEF DESCRIPTION OF THE SEQUENCES

SEQ ID NO:1 corresponds to an amino acid sequence of mature ovine interferon-τ (IFNτ; oTP-1; GenBank Accession No. Y00287; PID g1358).

SEQ ID NO:2 corresponds to an amino acid sequence of mature ovine IFNτ, where the amino acid residues at positions 5 and 6 of the sequence are modified relative to the sequence of SEQ ID NO:1.

### DETAILED DESCRIPTION

I. Definitions

Interferon-tau, abbreviated as IFNτ or interferon-τ, refers to a protein having greater than 70% amino acid homology to known IFNτ sequences (e.g., Ott, et al., J. Interferon Res., 11:357 (1991); Helmer, et al., J. Reprod. Fert., 79:83 (1987); Imakawa, et al., Mol. Endocrinol, 3:127 (1989); Whaley, et al., J. Biol. Chem., 269:10846 (1994); Bazer, et al., WO 94/10313 (1994)). Amino acid homology can be determined using, for example, the LALIGN program with default parameters. This program is found in the FASTA version 1.7 suite of sequence comparison programs (Pearson and Lipman, PNAS, 85:2444 (1988); Pearson, Methods in Enzymology, 183:63 (1990); program available from William R. Pearson, Department of Biological Chemistry, Box 440, Jordan Hall, Charlottesville, Va.). IFNτ sequences have been identified in various ruminant species, including but not limited to, cow (Bovine sp., Helmer S. D., J. Reprod. Fert., 79:83 (1987); Imakawa, K., Mol. Endocrinol., 119:532 (1988)), sheep (Ovine sp.), musk ox (Ovibos sp.), giraffe (Giraffa sp., GenBank Accession no. U55050), horse (Equus caballus), zebra (Equus burchelli, GenBank Accession no. NC005027), hippopotamus (Hippopotamus sp.), elephant (Loxodonta sp.), llama (Llama glama), goat (Capra sp., GenBank Accession nos. AY357336, AY357335, AY347334, AY357333, AY357332, AY357331, AY357330, AY357329, AY357328, AY357327), and deer (Cervidae sp.). The nucleotide sequences of IFNτ for many of these species are reported in public databases and/or in the literature (see, for example, Roberts, R. M. et al., J. Interferon and Cytokine Res., 18:805 (1998), Leaman D. W. et al., J. Interferon Res., 12:1 (1993), Ryan, A. M. et al., Anim. Genet., 34:9 (1996)). The term “interferon-tau” intends to encompass the interferon-tau protein from any ruminant species, exemplified by those recited above. In a preferred embodiment, the interferon-tau protein has at least about 80%, preferably 85%, more preferably 90%, still more preferably 95% sequence identity to one of the aforementioned interferon-tau sequences, and in a preferred embodiment to ovine interferon-tau.

Ovine IFNτ (OvIFNτ) refers to a protein having the amino acid sequence as identified herein as SEQ ID NO:1, and to proteins having amino acid substitutions and alterations such as neutral amino acid substitutions that do not significantly affect the activity of the protein, such as the IFNτ protein identified herein as SEQ ID NO:2. More generally, an ovine IFNτ protein is one having about 80%, more preferably 90%, still more preferably 95%, sequence homology to the sequence identified as SEQ ID NO:1. Sequence homology is determined, for example, by a strict amino acid comparison or using one of the many programs commercially available.

II. Treatment Methods

A treatment method for diminishing the formation of scar tissue at the site of a wound, and for improving the appearance of scar tissue as the tissue forms at a wound site, are described. Moreover, methods for preventing excessive scar formation and adhesions are described. These methods are achieved by administering locally to the site of the wound or by administering systemically an effective amount of IFNτ.

As discussed above, human skin when wounded does not the regenerate, but undergoes a repair process where connective tissue is deposited, forming a scar at the wound site. Applicant has found that administration of IFNτ to the subject before or during the wound healing process results in a reduction of scar tissue formation. As previously described in, for example U.S. Publication No. 2005-0142109 A1, orally administered IFNτ is effective to increase the concentration of circulating interleukin-10 (IL-10), an anti-inflammatory cytokine. Example 1 presents a study where IFNτ was orally administered to human subjects suffering from multiple-sclerosis. The blood level of IL-10 was measured as a function of time in the patients, and was observed to increase as a function of dose.

An increased level of IL-10 when administered to subjects having a tissue injury is shown to result in a reduction of formation of scar tissue. IFNτ applied locally to the wound or administered systemically to the patient increases the IL-10 level locally and/or systemically, to diminish the scar tissue formation, as described in Examples 2 and 3. At the same time, the wound healing process proceeds normally; that is, the wound closes and heals as expected, other than the extent of scar tissue formation.

IFNτ is a type I IFN first identified as a pregnancy recognition hormone in ruminants, such as sheep and cows (Bazer, F. W. et al., Am. J. Reprod. Immunol. 26:19-22 (1991)). The protein possesses antiviral and anti-proliferative properties, with considerably lower toxicity than other type I interferons (Pontzer, C., et al., Biochem. Biophys. Res. Comm., 152(2):801-807 (1988); Pontzer, C., et al., Cancer Res., 51:5304 (1991)). Relative to other interferons, ovine IFNτ shares about 45-55% identity with IFN-αs from human, mouse, rat, and pig and 70% homology with bovine IFN-αll, now referred to as IFN-Ω. A cDNA of ovine IFNτ and several cDNA sequences which may represent different isoforms have been reported in the literature (Imakawa, K. et al., Nature, 330:377-379, (1987); Stewart, H. J., et al., Mol. Endocrinol. 2:65 (1989); Klemann, S. W., et al., Nuc. Acids Res. 18:6724(1990); and Charlier, M., et al., Mol. Cell Endocrinol. 76:161-171 (1991)). All are approximately 1 kb with a 585 base open reading frame that codes for a 23 amino acid leader sequence and a 172 amino acid mature protein.

The 172 amino acid sequence of ovine-IFNτ is set forth, for example, in U.S. Pat. No. 5,958,402, and its homologous bovine-IFNτ sequence is described, for example, in Helmer et al., J. Reprod. Fert., 79:83-91 (1987) and Imakawa, K. et al., Mol. Endocrinol., 3:127 (1989). The sequences of ovine-IFNτ and bovine-IFNτ from these references are hereby incorporated by reference. An amino acid sequence of ovine IFNτ is shown herein as SEQ ID NO:1. A modified amino acid sequence of ovine IFNτ is shown herein as SEQ ID NO:2.

Recombinant production of IFNτ is described in both the scientific literature (Ott, et al., J. Interferon Cytokine Res., 11:357-364 (1991); Soos, J. M. et al., J. Immunol., 155:2747 (1995)) and the patent literature (WO/94/10313; US 2003/0049277, the description of IFNτ production in these documents is incorporated by reference herein.)

Exemplary Compositions for Administration

Pharmaceutical compositions containing IFNτ are prepared based on the specific desired route of administration. Compositions for topical, localized, or systemic administration are described herein.

Compositions for local or systemic administration will generally include an inert diluent. Solutions or suspensions used for parenteral, intradermal, subcutaneous, or topical application can include one or more of the following components: a sterile diluent such as water for injection, saline solution, oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, histidine, citrates, or phosphates; and agents for the adjustment of tonicity such as sodium chloride or dextrose. A parental preparation can be enclosed in ampoules, disposable syringes, or multiple dose vials made of glass or plastic.

Systemic compositions can be delivered either parenterally, mucosally, or enterally. Parenteral administration can be achieved by injection or by placement, via injection or via a catheter, of a depot using a controlled or sustained release formulation. In a preferred systemic composition, the IFNτ is formulated for oral administration, as described for example in U.S. Pat. Nos. 6,372,206 and 5,906,816. In one embodiment, the compositions for oral administration are formulated in an enteric carrier to protect the drug during passage through the stomach. Orally administrable preparations can be in the form of a tablet or capsule, and will generally include conventional tableting ingredients, such as a binder, such as microcrystalline cellulose, gum tragacanth or gelatin; an excipient such as starch or lactose; a disintegrating agent such as alginic acid or corn starch; a lubricant such as magnesium stearate; or a glidant such as colloidal silicon dioxide. When the dosage unit form is a capsule, it can contain, in addition to any of the materials of the above type, a liquid carrier such as a fatty oil. In addition, dosage unit forms can contain various other materials which modify the physical form of the dosage unit, for example, coatings of sugar, shellac, or other enteric agents. Mucosal administration can include formulations suitable for intranasal, buccal, or vaginal delivery.

Compositions of IFNτ for local or topical application can be formulated in a carrier such as saline or PBS, in an ointment or gel, in a transdermal patch or bandage, or in a controlled or sustained release formulation. Local administration can be by injection at the site of the injury, or by spraying topically onto the injury. The IFNτ can be absorbed into a bandage for direct application to the wound, or released from sutures or staples at the site. For topical or local application, IFNτ can be incorporated into a carrier in the form of an ointment, cream, gel, paste, foam, aerosol, suppository, pad or gelled stick. For example, a topical ointment or gel composition might consist of an effective amount of IFNτ in an excipient such as a mineral oil or a vegetable oil, or petroleum jelly, with a viscosity enhancing agent.

Any of these compositions may also include preservatives, antioxidants, antibiotics, immunosuppressants, and other biologically or pharmaceutically effective agents which do not exert a detrimental effect on the normal tissue to be treated.

It will be appreciated that the methods described herein can be conducted alone or in combination with other treatments. For example, administration of IFNτ can be administered in combination with antibiotics, cytokines, antiviral drugs, anti-inflammatory agents, or the like. Other combinations will be apparent to those skilled in the art.

Methods of Use

The IFNτ compositions are administered to a subject having a wound, in order to reduce scar formation and/or to prevent excessive scar formation, especially hypertrophic scars and keloid scars, and adhesions, especially intra-peritoneal or pelvic adhesions such as those resulting after open or laproscopic surgery, and burn contractions. As used herein “excessive scar formation” refers to the formation of scar tissue that is characterized by one of more of (i) widened or unsightly, but does not necessarily extend beyond the original boundaries of the wound; (ii) grows beyond the boundary of the initial injury, (iii) is raised beyond the plane of the skin. Other wounds which can be beneficially treated using the IFNτ compositions include prevention of scarring following transplantation, implantation of temporary prosthetics, and adhesions after surgery.

The IFNτ composition will preferably be administered either at the time of injury or surgery, or shortly thereafter. A medical provider can provide guidance regarding dosing regimen, depending on the location and severity of the wound. For example, a minor epidermal or dermal abrasion or laceration may be treated by topical application of IFNτ after there has been initial re-epithelialization of the skin's surface wounds, generally within several days after injury. A minor epidermal or dermal abrasion or laceration could also be treated with a systemic dose of IFNτ provided shortly after injury. In the case of adhesions, therapy will start early, that is, soon after procedures which lead to local trauma and the deposition of a transitional matrix.

The IFNτ composition is administered in a dosage and in a regimen that does not prevent wound healing, but does result in an increase in IL-10 locally or systemically, to decrease or prevent formation of connective tissue that leads to scar formation. Dosages will typically be in the same range as used for inhibition of viral growth or cellular proliferation, but administered to a different class of patients and for different time periods, since wound healing typically occurs over a much shorter time. When administered topically or in a sustained release formulation, the dosage of IFNτ may be considerably lower than, for example, an oral dosage. Selection of a suitable dosage can be made by a skilled medical provider. Selection of a suitable dosage can also be discerned by evaluating IL-10 blood levels using, for example and ELISA assay test kit, to monitor changes in IL-resulting from administration of IFNτ.

The specific activity of IFNτ may vary depending on the method of manufacture, but is readily measured using using a standard cytopathic effect assay (Familletti, P. C., et al., Methods in Enzymology, 78:387-394 (1981); Rubinstein, S. et al., J. Virol., 37:755-758 (1981)). Briefly, dilutions of IFNτ are incubated with Madin-Darby bovine kidney (MDBK) cells for 16-18 hours at 37° C. Following incubation, inhibition of viral replication is determined in a cytopathic effect assay using vesicular stomatitis virus as challenge. One antiviral unit (U) causes a 50% reduction in destruction of the monolayer. IFNτ generally has a specific activity of about 1×10⁸ antiviral U/mg protein. In one embodiment, a topical dose of IFNτ is from between 100-10,000 U/day. In another embodiment, a dose of between about 1×10³ to 1×10⁹ U/day is provided. These dosages are merely exemplary, and as noted above, a suitable dosage can be ascertained by a skilled caregiver.

### EXAMPLES

The following examples are illustrative in nature and are in no way intended to be limiting.

### Example 1

### Increase in IL-10 Concentration

IFNτ increases IL-10 concentrations in humans, which results in a reduction in scar tissue formation, as demonstrated by the following data. Humans suffering from multiple sclerosis were enrolled in a trial for treatment with IFNτ. Fifteen patients were randomized into three treatment groups: Group I patients were given IFNτ orally at a dosage of 0.2 mg per day (2×10⁷ U/day) Group II patients were given IFNτ orally at a dosage of 0.8 mg per day (8×10⁷ U/day); and Group III patients were given IFNτ orally at a dosage of 1.8 mg per day (1.8×10⁸ U/day).

TABLE A

| | Group I | Group II | Group III |
|---|---|---|---|
| | (n = 5) | (n = 5) | (n = 5) |
| IFNτ Oral Dose¹ | 0.2 mg/day | 0.6 mg/day | 1.8 mg/day |
| | (2 × 10⁷ U) | (6 × 10⁷ U) | (1.8 × 10⁸ U) |
| Average Weight | 67.2 kg | 58.9 kg | 90.0 kg |
| Average Age | 30 | 34.5 | 47 |

¹1 mg IFNτ = 1 × 10⁸ Units

Prior to treatment with IFNτ, on screening Day and Day 1 (one), a blood sample was taken from each subject to determine a baseline serum cytokine concentration. Treatment was initiated by administering IFNτ orally to each patient following the blood draw on Day 1. Prior to administration, the vials of IFNτ (SEQ ID NO:3) and syringes were kept in a refrigerator maintained at 2 to 8° C. Prior to self-administration of medication, the patient removed one vial and one syringe from the refrigerator. The cap was removed from the tip of the syringe and the tip of the syringe was placed into the bottle of medication to withdraw the appropriate volume into the syringe as instructed at the clinic on Day 1. The tip of the syringe was placed in the mouth and the syringe contents were emptied into the mouth by depressing the plunger. The patient then swallowed, and if desired, was allowed to drink a glass of water. The patient noted on his/her diary card the date and time the dose was administered.

Blood samples were taken from each patient on Days 1, 4, 8, 15, 29, and 57 of the study. The samples were analyzed for IL-10 concentrations and IFN-γ concentrations by using commercially available ELISA kits (Genzyme, Cambridge, Mass.).

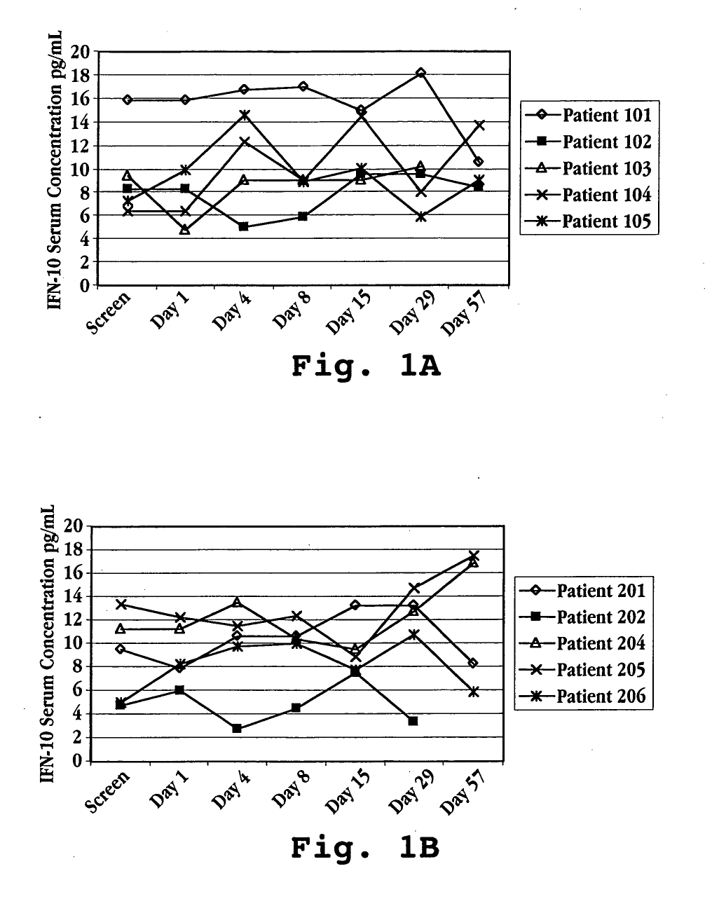

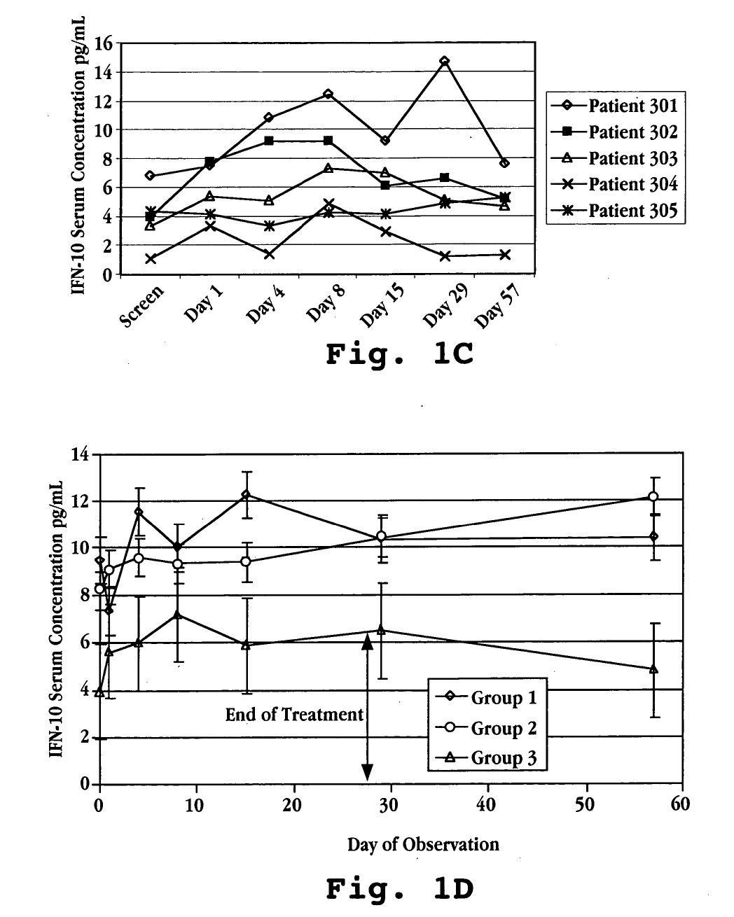

The IL-10 levels for the patients in Groups I, II, and III are shown in FIGS. 1A-1C, respectively. FIG. 1A shows serum IL-10 levels, in pm/mL, for the five patients in Group I. Three of the patients, patient numbers 103, 104, and 105, showed an increase in IL-10 level at Day 4, however the IL-10 levels decreased on the Day 8 reading in these patients. The IL-10 levels at Days 8 and 15 in Patient nos. 103 and 104 were not significantly changed from the level at Day 4. FIGS. 1B and 1C show the results for the patients in test Groups II and III, respectively. There is a suggestion of a slight increase in serum IL-10 levels after administration of IFNτ, particularly in the Group III patients. FIG. 1D shows the mean IL-10 serum levels, in pg/mL, for Groups I, II, and III. A slight upregulation of IL-10 in the test groups during the period of IFNτ dosing, between Days 2 and 28, however, the slight upregulation was not statistically significant, based on the statistical analysis set forth in Example 1. The small increase in IL-10 blood level continued in Groups I and II for a period of time after dosing with IFNτ was stopped on Day 28. The IL-10 serum levels at Day 57, which is 34 days after the last dose of IFNτ, remained above the baseline levels measured on Day 0 and Day 1.

### Example 2

### Inhibition of Scar Formation

Full thickness mouse wounds are made in adult mice, ranging in age from six weeks to sixteen weeks. Mice are treated daily with IFNτ administered topically to the wound site. Other mice are left untreated. The wounds are inspected daily, and at days 7, 14, and 21 post injury, histological micrographs of open mouse wounds are taken. Tissue biopsies taken at these time points are fixed, embedded, sectioned and stained with hematoxylin and eosin. Mice treated with IFNτ show a reduction in scar tissue formation.

### Example 3

### Inhibition of Scarring During Wound Healing

Mice are treated essentially the same as described in Example 2, however, prior to injury, mice are pretreated for ten days with oral administration of IFNτ added to the feed. A separate group of control mice were left untreated before and after injury. One month after injury, the resulting scars were examined by histological analysis, in the control and treated mice. Mice treated with IFNτ show a reduced scar tissue thickness compared to the thickness of the scar formed in untreated mice.

### Example 4

### Reduced Scar Tissue Formation in Human

A young male, age 17, presents with two lacerations on the right forearm. After cleaning the wound, and prior to suturing, a gel containing IFNτ is applied to one of the two lacerations designated for INFτ treatment. The wounds are then sutured closed. For the following four weeks, the IFNτ gel is topically applied to the wound designated for treatment two to three times per day. After four weeks, a visual inspection of the wounds reveals that the wound treated with IFNτ topical gel has significantly less scar tissue than the untreated, adjacent wound.

### Example 5

### Prevention of Excessive Scar Tissue Formation in a Human

A young female, age 27, with a history of keloid scarring, presents with a laceration on her left upper leg. After cleaning the wound, and prior to suturing, a gel containing IFNτ is applied to the wound. The wound is then sutured closed. For the following four weeks, the IFNτ gel is topically applied to the wound three to four times per day. In addition, the woman takes a daily oral dose of 1×10⁵ U of IFNτ. After four weeks, a visual inspection of the wound reveals that the wound healed with no keloid scar tissue formation.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed:

1. A method for reducing formation of scar tissue, comprising administering an effective amount of interferon-tau.

2. The method of claim 1, wherein the interferon-tau is administered topically.

3. The method of claim 2, wherein said topical administration is achieved by applying a paste, a cream, a suspension, or an ointment to a wound.

4. The method of claim 1, wherein the interferon-tau is administered systemically.

5. The method of claim 4, wherein said systemic administration is achieved via oral or mucosal administration.

6. The method of claim 1, wherein said interferon-tau has a sequence at least 80% identical to SEQ ID NO:2.

7. The method of claim 1, wherein said interferon-tau has a sequence identified herein as SEQ ID NO:1 or SEQ ID NO:2.

8. The method of claim 1, wherein said interferon-tau is formulated in a preparation comprising histidine.

9. The method of claim 1, further including pre-treating a subject with interferon-tau prior to wound formation.

10. A method for preventing excessive scar tissue formation, comprising administering an effective amount of interferon-tau.

11. The method of claim 10, wherein said administering is via topical or local delivery.

12. The method of claim 11, wherein said interferon-tau is administered by subcutaneous injection to a wound site.

13. The method of claim 11, wherein said interferon-tau is administered by topical application of a paste, ointment, or cream to a wound site.

14. The method of claim 10, wherein said interferon-tau is administered systemically.

15. The method of claim 14, wherein said interferon-tau is administered orally or mucosally.
